(19) United States
(12) Patent Application Publication
(10) Pub. No.: US 2008/0292702 A1
(43) Pub. Date: Nov. 27, 2008

(54) Solid Dispersion Comprising An Active Ingredient Having A Low Melting Point And Tablet For Oral Administration Comprising Same

(75) Inventors: Jong Soo Woo, Suwon-si (KR); Sang Wook Kim, Suwon-si (KR); Hong Gi Yi, Suwon-si (KR); Jae Kuk Ryu, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: Hanmi Pharm. Co. Ltd., Hwaseong-gun, Kyungki-do (KR)

(21) Appl. No.: 12/097,299

(22) PCT Filed: Dec. 18, 2006
(86) PCT No.: PCT/KR2006/005526

§ 371 Date: Jun. 13, 2008

(30) Foreign Application Priority Data
Dec. 16, 2005 (KR) 10-2005-0124386

(51) Int. Cl. A61K 9/24; A61K 31/192; A61K 47/38; A61K 47/36; A61K 47/02; A61K 47/32; A61K 9/22

(52) U.S. Cl. 424/472; 514/570; 514/781; 514/778; 514/770; 514/769; 514/772.5; 424/468

(57) ABSTRACT

A fused solid dispersion comprising an active ingredient having a melting point of 800 C or below and a pharmaceutically acceptable absorbent having a specific surface area ranging from 20 to 400 mVg can be conveniently compressed into a tablet without generating capping and sticking problems, and a tablet comprising said fused solid dispersion can maintain an uniform release rate over a prolonged time when orally administered.

### FIELD OF THE INVENTION

The present invention relates to a compress tabletting fused solid dispersion comprising an active ingredient having a low melting point, and a tablet for oral administration comprising same.

### BACKGROUND OF THE INVENTION

Non steroidal anti-inflammatory drugs such as ibuprofen or dexibuprofen (S(+)-ibuprofen) having low melting points, tend to melt by the heat generated during a compress tabletting process, causing the problems of capping and sticking, particularly when the drug content is high. In order to prevent such problems, a relatively high amount of excipient needs to be used but, in this case, the dosage unit must be increased to achieve an effective plasma concentration of the active ingredient.

Accordingly, there have been reported numerous methods for effectively compressing such a low-melting active ingredient into a tablet. For example, WO 92/020334 and DE 3,922,441 disclose a pharmaceutical composition comprising an ibuprofen or dexibuprofen salt, and WO 93/004676 discloses a pharmaceutical composition comprising ibuprofen agglomerates using a suspension comprising ibuprofen or a salt thereof, starch, surfactant, water and a solvent.

WO 95/001781 discloses a method for preparing a bilayer tablet consisting of a rapid release layer and a controlled release layer, wherein the rapid release layer comprises ibuprofen, corn starch, cross-linked polyvinylpyrrolidone, carboxymethyl starch, magnesium stearate, while the controlled release layer comprises ibuprofen, mannitol, hydroxypropylmethyl cellulose, talc, magnesium stearate and colloidal silica.

However, the above methods are not to fully satisfactory in solving the problems of capping and sticking that occur during a compression tabletting process.

### SUMMARY OF THE INVENTION

Accordingly, it is an object of the present invention to provide a fused solid dispersion comprising an active ingredient having a low melting point which can be easily compressed into a tablet.

It is another object of the present invention to provide a tablet for oral administration comprising same, which is capable of maintaining uniform release of the drugs over a long period of time.

It is still another object to provide a method for the preparation of said tablet.

In accordance with one aspect of the present invention, there is provided a fused solid dispersion comprising an active ingredient having a melting point of 80° C. or below and a pharmaceutically acceptable absorbent having a specific surface area ranging from 20 to 400 m²/g.

In accordance with one aspect of the present invention, there is provided a controlled release tablet for oral administration comprising the fused solid dispersion.

In accordance with another aspect of the present invention, there is provided a multilayer tablet for oral administration consisting of a rapid release layer and a controlled release layer containing the fused solid dispersion.

In accordance with still another aspect of the present invention, there is provided a process for preparing a tablet for oral administration comprising:

(a) heating to melt an active ingredient having a melting point of 80° C. or below and adding a pharmaceutically acceptable absorbent having a specific surface area ranging from 20 to 400 m²/g thereto to obtain a homogenous fused solid dispersion;

(b) cooling, drying and pulverizing the fused solid dispersion obtained in step (a) to obtain granules; and

(c) adding a release-controlling agent or a pharmaceutically acceptable excipient to the granules obtained in step (b) and compressing the resulting mixture into a tablet.

### BRIEF DESCRIPTION OF DRAWINGS

FIG. 1: the in vitro dissolution profiles of the rapid release tablets prepared in Examples 6 to 10 of the present invention;

FIG. 2: the in vitro dissolution profiles of the controlled release tablet prepared in Example 11 of the present invention as well as those of the bilayer tablets consisting of a rapid release layer and a controlled release layer prepared in Examples 12 and 13 of the present invention;

FIG. 3: the variation in the in vitro dissolution profile of the tablet prepared in Example 12 of the present invention as function of the rate of the paddle rotation;

FIG. 4: the in vitro dissolution profiles of the bilayer tablets consisting of a rapid release layer and a controlled release layer prepared in Examples 14 to 16 of the present invention; and

FIG. 5: the variation in the in vitro dissolution profile of the tablet prepared in Example 16 of the present invention as function of the rate of the paddle rotation.

### DETAILED DESCRIPTION OF THE INVENTION

The inventive tablet for oral administration comprises a controlled release tablet comprising a fused solid dispersion containing an active ingredient and a release-controlling agent, a rapid release tablet comprising the fused solid dispersion and a pharmaceutically acceptable excipient, and a multilayer tablet for oral administration having a controlled release layer formed using ingredients for the controlled release tablet and a rapid release layer, using ingredients for the rapid release tablet.

Each ingredient of the inventive tablet is described in detail as follows:

### <Fused Solid Dispersion>

The fused solid dispersion of the present invention comprises an active ingredient having a melting point of 80° C. or below and one or more pharmaceutically acceptable absorbent having a specific surface area ranging from 20 to 400 m²/g. The fused solid dispersion may further comprise a tabletting aid selected from the group consisting of a sugar alcohol, a water soluble polymer, an oily base and a mixture thereof. The weight ratio of the active ingredient:the pharmaceutically acceptable absorbent:the tabletting aid preferably ranges from 1:0.01˜3:1˜2.

### (1) Active Ingredient

In the present invention, the active ingredient used in the fused solid dispersion has a melting point of 80° C. or below, preferably 50 to 80° C., and representative examples of the active ingredient include ibuprofen (melting point: 75˜77° C.), dexibuprofen (melting point: 50˜54° C.) or a mixture thereof which are non steroidal anti-inflammatory drugs useful in the treatment of a rheumatoid arthritis

### (2) Pharmaceutically Acceptable Absorbent

In the present invention, the pharmaceutically acceptable absorbent used in the fused solid dispersion may be any of those conventionally used in the pharmaceutical field, and representative examples of the absorbent include light anhydrous silicic acid, hydrotalcite, aluminum magnesium silicate, aluminum hydroxide, aluminum silicate, magnesium aluminum methasilicate, bentonite, lactose, dextrin, starch, microcrystalline cellulose, hydroxypropylmethyl cellulose, hydroxypropyl cellulose, hydroxyethyl cellulose, ethyl cellulose, methyl cellulose, polyethylene glycol, finely-divided cross-linked polyvinylpyrrolidone or a mixture thereof.

Particularly, in order to avoid the problems such as capping and sticking occurring due to melting of the active ingredient by the heat generated during compression tabletting, a pharmaceutically acceptable absorbent having a specific surface area ranging from 20 to 400 m²/g, preferably, 100 to 300 m²/g, more preferably, 150 to 250 m²/g is used. When the range of the specific surface area of pharmaceutically acceptable absorbent is less than the lower limit, the capping and sticking problems still occur. In accordance with the present invention, the weight ratio of the active ingredient and the absorbent may range from 1:0.01˜3, preferably, from 1:0.1˜2. The absorbent may be added after heating to melt the active ingredient.

### (3) Tabletting Aid (Sugar Alcohol, Water Soluble Polymer, Oily Base or Mixture Thereof)

In order to facilitate the granulation after grinding and increase the binding efficiency of the granules during compress tabletting by way of diminishing the melting fixation, the inventive fused solid dispersion may further comprise a tabletting aid selected from the group consisting of a sugar alcohol, a water soluble polymer, an oily base and a mixture thereof. The weight ratio of the active ingredient:the tabletting aid preferably ranges 1:0˜2.

Representative examples of the sugar alcohol used in the present invention include xylitol, sorbitol, mannitol and a mixture thereof, representative examples of the water soluble polymer include hydroxypropylmethyl cellulose, hydroxypropyl cellulose, polyvinylpyrrolidone, polyethylene glycol, polyethylene oxide, polyvinyl alcohol and a mixture thereof, and the representative examples of the oily base include sucrose fatty acid ester, glyceryl behenate, glyceryl palmitostearate, glyceryl monooleate, glyceryl monostearate and a mixture thereof.

The fused solid dispersion according to the present invention may be prepared using any conventional mixer, preferably a universal mixer or a heat-melt extruder.

The method of preparing the fused solid dispersion with a universal mixer or the heat-melt extruder is described in detail as follows:

### (a) Preparation of the Fused Solid Dispersion Using a Universal Mixer

The active ingredient is added to a universal mixer preheated to 60° C. to 100° C. and heat-melted, followed by mixing homogeneously. A pharmaceutically acceptable absorbent having a specific surface area ranging from 20 to 400 m²/g is added to the melten drug and the mixture is stirred for 20 to 60 minutes to obtain a homogeneous dispersion. At this time, a tabletting aid such as a sugar alcohol, a water soluble polymer and an oily base may be further added the dispersion. After shutting down the heater, the dispersion is stirred at room temperature, and the resulting agglomerate is collected and dried by cold blasting to obtain a fused solid dispersion comprising the active ingredient. The fused solid dispersion thus obtained is ground with a high-speed grinder and the resulting granules are filtered through No. 14 mesh (1410 μm) to 20 mesh (850 μm), preferably 20 mesh (850 μm) to obtain a fused solid dispersion.

(b) Preparation of the Fused Solid Dispersion with a Heat-Melt Extruder

The active ingredient and the pharmaceutically acceptable absorbent having a specific surface area ranging from 20 to 400 m²/g are homogeneously mixed, and the mixture placed in a loading hopper is heated to melt in the hot compression screw chamber, followed of extruding the melt. The obtained agglomerate is homogeneously mixed with kneader-mixer, and the mixtures are filtered through a screen to obtain a fused solid dispersion having a uniform size.

In this process, the length of the time the active ingredient is exposed to the heat is shortened, and a fused solid dispersion having a uniform size distribution can be obtained. Thus, a fused solid dispersion having a uniform size distribution can be manufactured by a less time-consuming single process which is conducted by carrying out the inputting, melting and screening of the active ingredients in sequence.

### <Tablet Comprising the Fused Solid Dispersion>

Various types of tablets such as controlled release tablet, rapid release tablet and multilayer tablet can be prepared by optionally adding a pharmaceutically acceptable excipient to the fused solid dispersion and compressing into a tablet without the use of a cooler. The compressed tablet preferably has a hardness in the range from 4 to 16 kp, preferably 8 to 12 kp.

### (A) Controlled Release Tablet

A controlled release tablet comprises the above-mentioned fused solid dispersion and a release-controlling agent and may further comprises a pharmaceutically acceptable excipient. The weight ratio of the fused solid dispersion:the release-controlling agent:the pharmaceutically acceptable excipient ranges from 1:0.01˜3:0˜3, and preferably, from 1:0.05˜2:0.01˜2.

### (A-1) Release-Controlling Agent

The release-controlling agent for maintaining uniform release rate for a long period of time can be selected from the group consisting of polyethylene oxide having a molecular weight ranging from 10,000 to 9,000,000, hydroxypropylmethyl cellulose having a molecular weight ranging from 1,000 to 4,000,000, hydroxypropyl cellulose, carboxyvinyl polymer, polyvinyl alcohol, xanthan gum, guar gum, locust bean gum, carboxymethyl cellulose and its derivative, methyl cellulose and its derivative, and povidone-polyvinylacetate copolymer having a molecular weight ranging from 2,000 to 2,000,000. In accordance with the present invention, the weight ratio of the fused solid dispersion release-controlling agent may range from 1:0.01˜3, and preferably, from 1:0.05˜2.

### (A-2) Pharmaceutically Acceptable Excipient

In the present invention, in order to maintain an appropriate hardness and dosage form of a tablet, the controlled release tablet may further comprise a pharmaceutically acceptable excipient.

The pharmaceutically acceptable excipient used in the present invention may be used any conventional one used in the pharmaceutical field, and representative examples of the pharmaceutically acceptable excipient include a cross-linked polyvinylpyrrolidone, a cross-linked sodium carboxymethyl cellulose, carboxymethyl starch, calcium methacrylate-divinylbenzene copolymer, polyvinyl alcohol, lactose, microcrystalline cellulose and cellulose derivative, starch and its derivative, cyclodextrin and dextrin derivative, pregelatinized starch and its derivative, colloidal silica, magnesium stearate, glyceryl monostearate, sodium stearyl fumarate, talc, and hydrogenated caster oil.

In accordance with the present invention, the weight ratio of the fused solid dispersion:the pharmaceutically acceptable excipient may range from 1:0˜3, and preferably, from 1:0.01˜2.

### (B) Rapid Release Tablet

In the present invention, a rapid release tablet comprises the above-mentioned fused solid dispersion, and the above-mentioned pharmaceutically acceptable excipient used in the controlled release tablet. The weight ratio of the fused solid dispersion:the pharmaceutically acceptable excipient may range from 1:0.05˜3, and preferably, from 1:0.1˜2.

### (C) Multilayer Tablet

A multilayer tablet in accordance with the present invention may be prepared by forming a controlled release layer with ingredients of the controlled release tablet and by forming a rapid release layer with ingredients of the rapid release tablet to manipulate the release of the active ingredient.

The bilayer tablet consisting of the rapid release tablet and the controlled release layer can be prepared by subjecting the ingredient for the rapid release layer to a first tablet compression step, depositing the ingredients for the controlled release layer thereon, and subjecting the resulting mixture to a second tablet compression step. The tablet compression process of the controlled release layer does not always have to be carried out after tabletting the rapid release layer. The tablet compression of the controlled release layer can be carried out first, and then the granules of the rapid release layer are added thereto, followed of tablet compression. Also the rapid release layer and the controlled release layer can be sequentially or reversely filled, which is compressed into a tablet in one step.

The multilayer tablet of the present invention can be also prepared as a trilayer tablet consisting of rapid release and controlled release layers.

When the tablet consisting of the rapid release layer and the controlled release layer comprising the same active ingredient according to the present invention is subjected to in vitro release tests in accordance with the paddle method at 100 rpm (Korea pharmacopoeia 8^{th} ed. in vitro dissolution tests 2^{nd} method) using 900 mL of artificial gastric fluid (Korea pharmacopoeia 8^{th} ed., the 2^{nd} solution for the disintegrating-test), 85% or more of the active ingredient of the rapid release layer preferably is released within about 1 hour after initiating the test, while the active ingredient of the controlled release layer is released sequentially, preferably in amounts corresponding to 1 to 30% within about 1 hour, 30 to 70% within about 5 hours, and 85% or more within 12 hours after initiating the test.

The span of the release time of the active ingredient of the controlled release layer or the controlled release tablet can be prolonged by controlling the type and amount of excipient used in the controlled release layer. When in vitro dissolution tests were conducted according to the above method, the active ingredient of the rapid release layer in the multilayer tablet is released, preferably in amounts corresponding to 1 to 30% within 1 hour, 30 to 70% within 6 hours, 60 to 90% within 12 hours, 80% or more within 24 hours after initiating the test.

The rapidly-released active ingredient allows the plasma drug concentration to promptly reach the effective treating level while the slowly-released active ingredient can maintain the effective plasma drug concentration during the intended time. Thus, the pharmaceutically useful tablet according to the present invention is easily prepared without being hindered by such problems as capping and sticking during the course of compression tabletting, which can be effectively implemented in a large-scale manufacturing process.

The following Examples are intended to further illustrate the present invention without limiting its scope.

### EXAMPLES

### Preparation of Fused Solid Dispersion

### Example 1

300 g of dexibuprofen was added to a universal mixer (VERSATILE MIXER (250DM-rrs), DALTON) preheated to 60° C. and was allowed to melt, followed by mixing homogeneously. 60 g of light anhydrous silicic acid having a specific surface area of 200±25 m²/g was slowly added thereto, and the mixture was stirred for 45 minutes to obtain a homogeneous dispersion (see Table 1). The resulting dispersion was cooled to the room temperature while stirring to obtain a solid dexibuprofen dispersion agglomeration. The resulting agglomeration was cooled to the room temperature by cold blasting (30° C.) for about 2 hours, and the resulting product was then ground with a high-speed grinder. The resulting granules were filtered through No. 20 mesh (850 μm) to obtain a fused solid dispersion.

### Example 2

A fused solid dispersion was prepared by repeating the procedure of Example 1 except for using 110 g of light anhydrous silicic acid having a specific surface area of 200±25 m²/g.

### Example 3

A fused solid dispersion was prepared by repeating the procedure of Example 1 except for using 110 g of light anhydrous silicic acid having a specific surface area of 300±25 m²/g.

### Example 4

A fused solid dispersion was prepared by repeating the procedure of Example 1 except that 300 g of dexibuprofen and 50 g of xylitol were added to the Universal mixer preheated to 95° C. and melted. 60 g of light anhydrous silicic acid having a specific surface area of 200±25 m²/g was slowly added thereto and the mixture was stirred for 45 minutes to obtain a homogeneous dispersion.

### Example 5

A fused solid dispersion was prepared by repeating the procedure of Example 4 except for using 20 g of hydroxypropylmethyl cellulose instead of 50 g of xylitol.

TABLE 1

| | Example 1 | Example 2 | Example 3 | Example 4 | Example 5 |
|---|---|---|---|---|---|
| Ingredients | (g) | (g) | (g) | (g) | (g) |
| Dexibuprofen | 300 | 300 | 300 | 300 | 300 |
| Light anhydrous Silicic acid (200 ± 25 m²/g) | 60 | 110 | — | 60 | 60 |
| Light anhydrous Silicic acid (200 ± 25 m²/g) | — | — | 110 | — | — |
| Xylitol | — | — | — | 50 | — |
| Hydroxypropyl methyl | — | — | — | — | 20 |
| cellulose | | | | | |
| Total | 360 | 410 | 410 | 410 | 380 |

### <Preparation of Rapid Release Tablet>

### Example 6

In accordance with the components listed in Table 2, 205 mg of the fused solid dispersion obtained in Example 3 (amount of dexibuprofen: 150 mg per tablet), 10 mg of lactose, 49.7 mg of microcrystalline cellulose, 3.8 mg of cross-linked sodium carboxymethyl cellulose, and 5.1 mg of light anhydrous silicic acid as a pharmaceutically acceptable excipient were mixed together for 60 minutes and 11.4 mg of talc as a lubricant was added thereto. The resulting mixture was stirred for 5 minutes and compressed to a hardness of about 8 to 12 kp to obtain a rectangular rapid release tablet.

### Examples 7 to 10

Fused solid dispersions were prepared by repeating the procedure of Example 6 using the component listed in Table 2.

TABLE 2

| | | Example 6 | Example 7 | Example 8 | Example 9 | Example 10 |
|---|---|---|---|---|---|---|
| Ingredients | | (mg) | (mg) | (mg) | (mg) | (mg) |
| Fused Solid dispersion | Example 2 | — | 205.0 | 136.7 | — | — |
| (amount of dexibuprofen) | | | (150) | (100) | | |
| | Example 3 | 205.0 | — | — | — | — |
| | | (150) | | | | |
| | Example 4 | — | — | — | 683.3 | — |
| | | | | | (500) | |
| | Example 5 | — | — | — | — | 380 |
| | | | | | | (300) |
| Excipient | Lactose | 10 | — | 6.7 | 33.4 | — |
| | Ludipress ® (BASF) | — | — | — | — | 95.0 |
| | Microcrystalline cellulose | 49.7 | — | 33.1 | 165.8 | — |
| | Hydroxypropyl cellulose | — | 3.8 | — | — | — |
| | Micro Shellac ® 100 (MEGLE) | — | 132 | — | — | — |
| | Cross-linked sodium | 3.8 | 36.2 | 2.5 | 12.5 | 20 |
| | Carboxymethyl cellulose | | | | | |
| | Light anhydrose Silicic acid | 5.1 | — | 3.4 | — | — |
| Lubricant | Magenesium stearate | — | 3.6 | — | 5.8 | 5 |
| | Talc | 11.4 | — | 7.6 | 24.2 | — |

| | | | | | |
|---|---|---|---|---|---|
| Total | 285 | 380.6 | 190 | 925 | 500 |

### Test Example 1

### In Vitro Dissolution Test of Rapid Release Tablet

The rapid release tablets prepared in Examples 6 to 10 were each subjected to an in vitro dissolution test based on Korea Food and Drug Administration (KFDA) and Release Guidelines on the drug for oral administration, and the release pattern was analyzed under the following conditions.

### <Dissolution Test Method>

Samples: Rapid release tablets prepared in Examples 6 to 10
Test solution: The disintegrating-test 2nd method described in Korea pharmacopoeia, pH 6.8 artificial gastric fluid, 900 mL, 37±0.5° C.
Dissolution method: The dissolution test method described in Korea pharmacopoeia (the paddle method), rotation speed: 50 rpm

TABLE 3

| | Dissolution rate (%) | | | | |
|---|---|---|---|---|---|
| Dissolution time(min) | Example 6 | Example 7 | Example 8 | Example 9 | Example 10 |
| 5 | 53.5 ± 4.0 | 62.8 ± 2.9 | 60.5 ± 1.5 | 59.0 ± 8.1 | 66.7 ± 2.6 |
| 10 | 69.9 ± 5.5 | 85.9 ± 1.3 | 80.6 ± 3.7 | 76.8 ± 1.7 | 89.1 ± 2.7 |
| 15 | 79.1 ± 4.8 | 93.7 ± 0.6 | 93.5 ± 2.1 | 91.6 ± 0.5 | 94.9 ± 1.1 |
| 30 | 94.7 ± 2.1 | 96.2 ± 0.6 | 98.5 ± 1.0 | 95.6 ± 4.9 | 95.6 ± 1.9 |
| 45 | 100.0 ± 0.3 | 98.9 ± 0.1 | 99.8 ± 1.0 | 100.8 ± 1.4 | 96.6 ± 2.2 |
| 60 | 101.1 ± 0.7 | 98.8 ± 0.1 | 100.5 ± 0.5 | 101.2 ± 2.4 | 97.6 ± 1.4 |

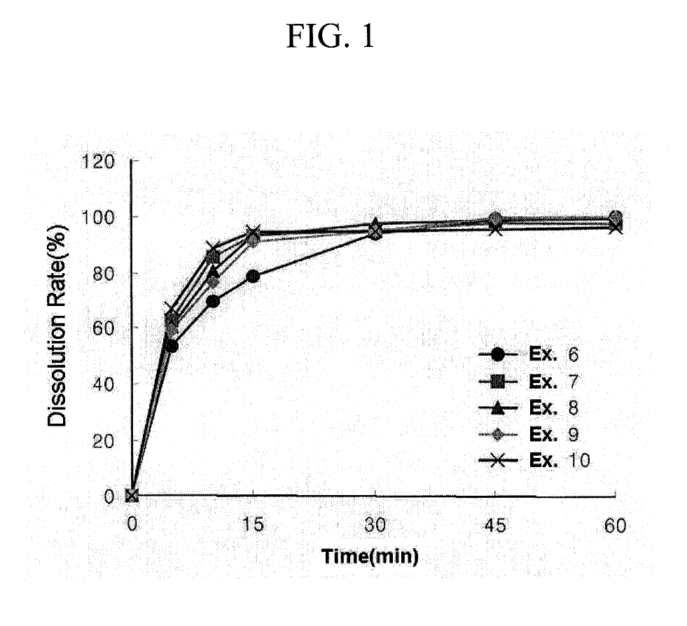

As can be seen from Table 3 and FIG. 1, each of the rapid release tablets prepared in Examples 6 to 10 showed a rapid drug release pattern (85% or more within 30 minutes after initiating release of the drug), and thus the inventive rapid release tablet comprising the inventive fused solid dispersion as an active ingredient provides rapid therapeutical effects.

### Example 11

### Preparation of Controlled Release Tablet

A controlled release tablet was prepared by repeating the procedure of Example 6 except that the fused solid dispersion, the release-controlling agent, and the lubricant listed in Table 4 were used.

TABLE 4

| Ingredients | | Example 11 |
|---|---|---|
| Fused solid dispersion | Example 5 | 231.8 |
| (amount of dexibuprofen) | | (183.0 mg) |
| Release-controlling agent | Hydroxypropylmethyl | 35.0 |
| | cellulose 2208, 4000SR | |
| | Calsium | 74.2 |
| | phosphate dibasic | |
| | Xanthangum | 28.0 |
| | Locust bean gum | 7.0 |
| | Micro shellac ® 100 | 30.0 |
| | Light anhydrose | 24.0 |
| | silicic acid | |
| Lubricant | Magnesium stearate | 4.8 |

| | |
|---|---|
| Total | 434.8 |

### Test Example 2

### In Vitro Dissolution Test of Controlled Release Tablet

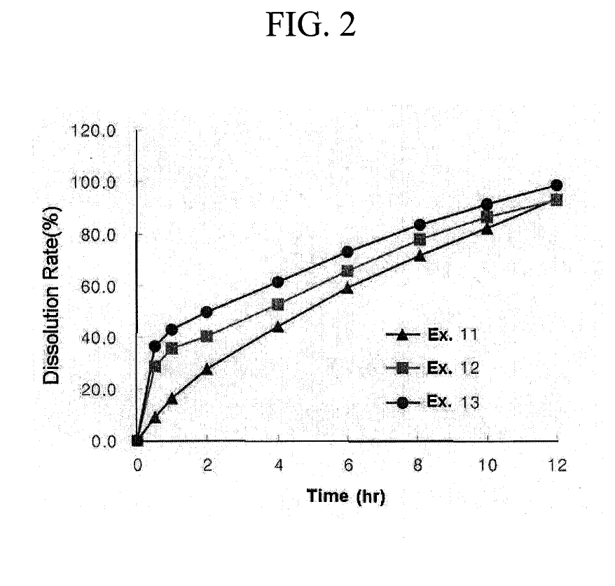

The controlled release tablet prepared in Example 11 was subjected to in vitro dissolution test under the following conditions, and the results are shown in Table 5 and FIG. 2.

### <Dissolution Test Method>

Sample: Controlled release tablet prepared in Example 11
Test solution: The disintegrating-test 2nd method described in Korea pharmacopoeia, pH 6.8 artificial gastric fluid, 900 mL, 37±0.5° C.
Dissolution method: the dissolution test method described in Korea pharmacopoeia (the paddle method), rotation speed: 100 rpm

TABLE 5

| Dissolution time(hr) | Dissolution rate(%) |
|---|---|
| 0.5 | 9.2 ± 1.2 |
| 1 | 16.5 ± 1.1 |
| 2 | 28.0 ± 1.1 |
| 4 | 44.3 ± 0.7 |
| 6 | 59.6 ± 1.1 |
| 8 | 72.0 ± 0.8 |
| 10 | 82.6 ± 2.4 |
| 12 | 93.9 ± 2.6 |

As can be seen in Table 5 and FIG. 2, the controlled release tablet prepared in Example 11 slowly released the active ingredient of the controlled release portion over a period of 12 hours.

### Examples 12 and 13

### Preparation of Bilayer Tablets Consisting of Rapid Release and Controlled Release Layers (1)

The components listed in Table 6 were mixed together and the mixture was subjected to a first tablet compression step to a hardness of about 2 to 3 kp, and then, the controlled release layer was deposited thereon and the resulting material was subjected to a second tablet compression step to a hardness of about 8 to 12 kp to obtain bilayer tablets.

TABLE 6

| | | | Exam- | Exam- |
|---|---|---|---|---|
| | | | ple | ple |
| | | | 12 | 13 |
| Ingredients | | | (mg) | (mg) |
| Rapid- | Resulting mixture of Example 7 | | 365.6 | — |
| release | (dexibuprefen: 150.0 mg) | | | |
| layer | Resulting mixture of Example 8 | | — | 190.0 |
| | (dexibuprefen: 100.0 mg) | | | |
| Con- | Fused solid | Example 2 | 478.3 | 239.2 |
| trolled- | dispersion | | (350.0 | (175.0 |
| release | (amount of | | mg) | mg) |
| layer | dexibuprefen) | | | |
| | Release- | Polyethylene oxide | 23.3 | 37.5 |
| | controlling | (Molecular weight: | | |
| | agent | 5,000,000) | | |
| | | Calsium phospate, | 76.1 | 36.2 |
| | | dibasic | | |
| | | Hydroxy propyl | 9.0 | 5.5 |
| | | cellalose | | |
| | Lubricant | Magnesium | 9.0 | — |
| | | stearate | | |
| | | Talc | — | 13.3 |

| | | |
|---|---|---|
| Total | 961.3 | 521.7 |

### Test Example 3

### In Vitro Dissolution Test of Bilayer Tablet (1)

In vitro dissolution tests were conducted using the bilayer tablets prepared in Examples 12 and 13 under the following condition, and the results are shown in Table 7 and FIG. 2.

### <Dissolution Test Method>

Samples: Bilayer tablets prepared in Examples 12 and 13
Test solution: The disintegrating-test 2nd method described in Korea pharmacopoeia, pH 6.8 artificial gastric fluid, 900 mL, 37±0.5° C.
Dissolution method: The dissolution test method described in Korea pharmacopoeia (the paddle method), rotation speed: 100 rpm

TABLE 7

Dissolution rate(%)

| Dissolution time(hr) | Example 12 | Example 13 |
|---|---|---|
| 0.5 | 28.6 ± 1.5 | 36.5 ± 2.8 |
| 1 | 35.7 ± 1.1 | 43.1 ± 2.4 |
| 2 | 40.5 ± 1.0 | 50.1 ± 2.2 |
| 4 | 53.0 ± 0.2 | 61.7 ± 2.2 |
| 6 | 66.1 ± 1.7 | 73.4 ± 2.0 |
| 8 | 78.2 ± 2.6 | 83.7 ± 0.9 |
| 10 | 87.1 ± 2.8 | 91.8 ± 1.1 |
| 12 | 93.4 ± 2.4 | 99.1 ± 0.2 |

As can be seen in Table 7 and FIG. 2, each of the bilayer tablets prepared in Examples 12 and 13 showed that all the active ingredient of the rapid release portion was released, regardless of the amount of the active ingredient, and thereafter, the active ingredient of the controlled release portion was slowly released over a period of 12 hours.

### Test Example 4

### In Vitro Dissolution Test of Bilayer Tablet (1) as Function of the Rotation Number

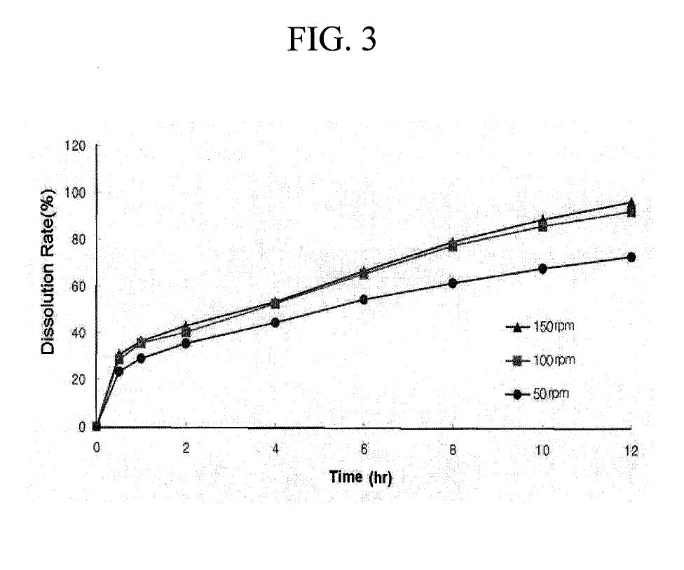

An in vitro dissolution test was conducted using the bilayer tablet prepared in Example 12 under the following conditions, and the results are shown in Table 8 and FIG. 3.

### <Dissolution Test Method>

Sample: Bilayer tablet prepared in Example 12
Test solution: The disintegrating-test 2nd method described in Korea pharmacopoeia, pH 6.8 artificial gastric fluid, 900 mL, 37±0.5° C.
Dissolution method: The dissolution test method described in Korea pharmacopoeia (the paddle method), rotation speed: 50, 100 and 150 rpm

TABLE 8

| | Dissolution rate (%) | | |
|---|---|---|---|
| | Revolution per minute(RPM) | | |
| Dissolution time(hr) | 50 rpm | 100 rpm | 150 rpm |
| 0.5 | 23.4 ± 0.9 | 28.6 ± 1.5 | 30.6 ± 0.3 |
| 1 | 28.8 ± 0.9 | 35.7 ± 1.1 | 36.2 ± 0.9 |
| 2 | 35.6 ± 0.2 | 40.5 ± 1.0 | 43.1 ± 2.0 |
| 4 | 44.9 ± 1.0 | 53.0 ± 0.2 | 54.0 ± 0.4 |
| 6 | 54.9 ± 2.3 | 66.1 ± 1.7 | 67.4 ± 0.3 |
| 8 | 62.4 ± 2.6 | 78.2 ± 2.6 | 80.2 ± 0.3 |
| 10 | 69.0 ± 2.7 | 87.1 ± 2.8 | 89.6 ± 0.2 |
| 12 | 74.4 ± 3.4 | 93.4 ± 2.4 | 97.5 ± 0.3 |

As can be seen from Table 8 and FIG. 3, the tablet rapidly released the drug during the initial 1 hour to provide prompt therapeutical effects, regardless of the rotation speed, and thereafter the tablet displayed a steady release pattern of the drug, suitable for maintaining continuous therapeutical effects.

### Examples 14 to 16

### Preparation of Bilayer Tablet (2)

The components of the controlled release listed in Table 9 were subjected to a first tablet compression step to a hardness of about 2 to 3 kp, and then, the rapid release layer was deposited thereon, and the resulting material was subjected to a second tablet compression step to a hardness of about 8 to 12 kp to obtain bilayer tablets.

TABLE 9

| | | | Example 14 | Example 15 | Example 16 |
|---|---|---|---|---|---|
| | | | (mg) | (mg) | (mg) |
| Rapid release | Resulting mixture of Example 8 | | 190.0 | 190.0 | 190.0 |
| layer | (dexibuprofen: 100.0 mg) | | | | |
| Controlled- | Fused solid dispersion | Example 2 | 239.2 | 239.2 | 239.2 |
| release layer | (amount of dexibuprofen) | (175.0 mg) | | | |
| | Release Controllng agent | Polyethylene oxide | 52.0 | 73.5 | 37.5 |
| | | (Molecular weight: 5,000,000) | | | |
| | | Xanthangum | — | — | 11.0 |
| | | Locust bean gum | — | — | 3.5 |
| | | Calsium phospate, dibasic | 36.2 | 36.2 | 36.2 |
| | | Hydroxypropyl cellulose | 5.5 | 5.5 | 5.5 |
| | Lubricant | Talc | 13.3 | 13.3 | 13.3 |

| | | | |
|---|---|---|---|
| Total | 536.2 | 557.7 | 536.2 |

### Test Example 5

### In Vitro Dissolution Test of Bilayer Tablet (2)

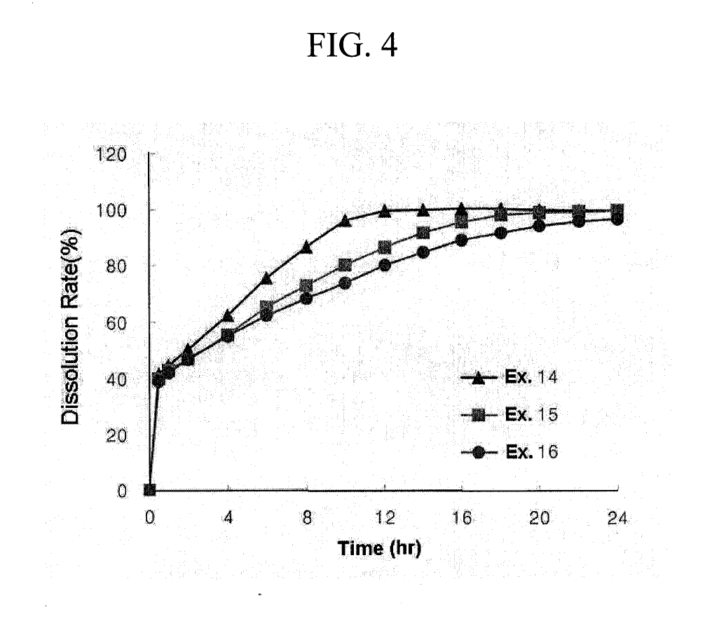

In vitro dissolution tests were conducted using the bilayer tablets prepared in Examples 14 to 16 under the following conditions, and the results are shown in Table 10 and FIG. 4.

### <Dissolution Test Method>

Samples: Bilayer tablets prepared in Examples 14 and 16
Test solution: The disintegrating-test 2nd method described in Korea pharmacopoeia, pH 6.8 artificial gastric fluid, 900 mL, 37±0.5° C.
Dissolution method: The dissolution test method described in Korea pharmacopoeia (the paddle method), rotation speed: 100 rpm

TABLE 10

| | Dissolution rate (%) | | |
|---|---|---|---|
| Dissolution time(hr) | Example 14 | Example 15 | Example 16 |
| 0.5 | 41.5 ± 1.6 | 39.9 ± 0.4 | 39.0 ± 0.4 |
| 1 | 44.8 ± 1.8 | 42.7 ± 0.6 | 42.1 ± 0.6 |
| 2 | 50.4 ± 1.9 | 46.6 ± 0.1 | 46.6 ± 0.7 |
| 4 | 62.6 ± 3.2 | 55.3 ± 1.1 | 54.8 ± 0.6 |
| 6 | 75.7 ± 5.6 | 65.4 ± 2.8 | 62.5 ± 0.7 |
| 8 | 87.2 ± 7.1 | 73.1 ± 3.7 | 68.5 ± 0.5 |
| 10 | 96.5 ± 5.1 | 80.6 ± 4.0 | 74.0 ± 0.1 |
| 12 | 100.2 ± 2.6 | 87.0 ± 3.6 | 80.5 ± 1.8 |
| 14 | 100.7 ± 2.1 | 92.5 ± 2.8 | 85.3 ± 1.9 |
| 16 | 100.9 ± 2.2 | 96.3 ± 2.0 | 89.7 ± 1.2 |
| 18 | 100.7 ± 2.0 | 98.6 ± 1.3 | 92.4 ± 0.8 |
| 20 | 100.5 ± 1.0 | 99.5 ± 0.5 | 95.0 ± 0.5 |
| 22 | 100.5 ± 1.9 | 99.9 ± 1.1 | 96.8 ± 1.2 |
| 24 | 100.6 ± 1.5 | 100.5 ± 0.9 | 97.5 ± 0.9 |

As can be seen from Table 10 and FIG. 4, all the active ingredient of rapid release layer was released within 1 hour, regardless of the amount of the active ingredient, and thereafter, the tablet released the active ingredient continuously for 12 to 24 hours.

### Test Example 6

### In Vitro Dissolution Test of Bilayer Tablet (2) as Function of the Rotation Number

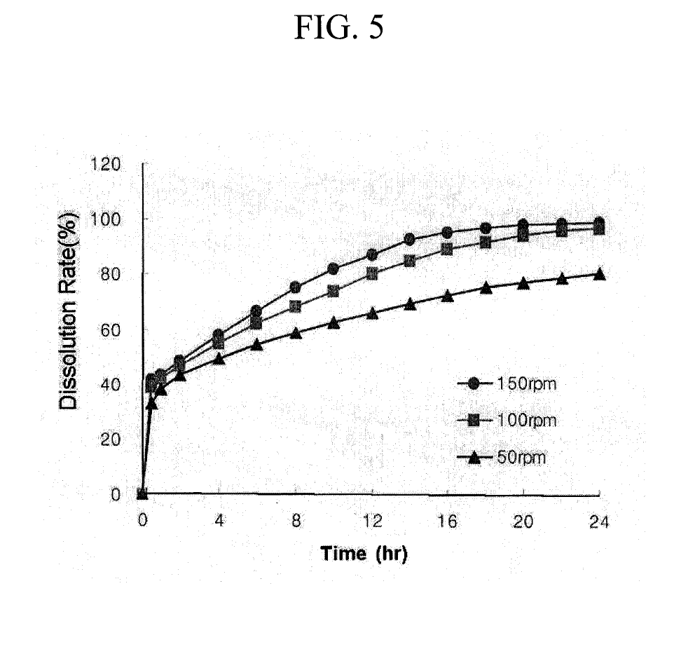

In vitro dissolution test was conducted using the bilayer tablet prepared in Example 16 under the following conditions, and the results are shown in Table 11 and FIG. 5.

### <Dissolution Test Method>

Sample: Bilayer tablet prepared in Example 16
Test solution: The disintegrating-test 2nd method described in Korea pharmacopoeia, pH 6.8 artificial gastric fluid, 900 mL, 37±0.5° C.
Dissolution method: The dissolution test method described in Korea pharmacopoeia (the paddle method), rotation speed: 50, 100 and 150 rpm

TABLE 11

| | Dissolution rate (%) | | |
|---|---|---|---|
| | Revolution per minute(RPM) | | |
| Dissolution time(hr) | 50 rpm | 100 rpm | 150 rpm |
| 0.5 | 32.9 ± 0.3 | 39.0 ± 0.4 | 41.6 ± 0.2 |
| 1 | 38.1 ± 1.4 | 42.1 ± 0.6 | 43.5 ± 0.5 |
| 2 | 43.4 ± 2.2 | 46.6 ± 0.7 | 48.5 ± 0.4 |
| 4 | 49.5 ± 2.7 | 54.8 ± 0.6 | 57.9 ± 1.9 |
| 6 | 54.5 ± 2.6 | 62.5 ± 0.7 | 66.7 ± 2.7 |
| 8 | 58.8 ± 2.8 | 68.5 ± 0.5 | 75.3 ± 3.8 |
| 10 | 62.7 ± 2.8 | 74.0 ± 0.1 | 82.4 ± 3.9 |
| 12 | 66.1 ± 2.8 | 80.5 ± 1.8 | 87.7 ± 4.1 |
| 14 | 69.6 ± 2.9 | 85.3 ± 1.9 | 93.0 ± 3.2 |
| 16 | 72.8 ± 2.9 | 89.7 ± 1.2 | 95.7 ± 2.4 |
| 18 | 75.6 ± 2.7 | 92.4 ± 0.8 | 97.3 ± 2.1 |
| 20 | 77.6 ± 1.5 | 95.0 ± 0.5 | 98.9 ± 1.1 |
| 22 | 79.2 ± 2.1 | 96.8 ± 1.2 | 99.2 ± 1.5 |
| 24 | 81.1 ± 0.9 | 97.5 ± 0.9 | 99.8 ± 0.5 |

As can be seen from Table 11 and FIG. 5, the tablet rapidly released the drug in the initial 1 hour to provide fast therapeutical effects, followed by a steady release pattern of the drugs suitable for continuously maintaining the therapeutical effect.

Accordingly, as shown from the result of the dissolution test, the active ingredient of the rapid release portion was rapidly released within the initial 1 hour to attain an effective blood concentration thereof, exerting fast therapeutical effects. The active ingredient of the controlled release portion was slowly released over a period of 12 to 24 hours, thereby maintaining an effective concentration of the drug in the blood at a constant level during the intended time.

While the embodiments of the subject invention have been described and illustrated, it is obvious that various changes and modifications can be made therein without departing from the spirit of the present invention which should be limited only by the scope of the appended claims.

1. A fused solid dispersion comprising an active ingredient having a melting point of 80° C. or below and a pharmaceutically acceptable absorbent having a specific surface area ranging from 20 to 400 m²/g.

2. The fused solid dispersion of claim 1, which comprises the active ingredient and the pharmaceutically acceptable absorbent in a weight ratio ranging from 1:0.01 to 1:3.

3. The fused solid dispersion of claim 1, wherein the active ingredient having a melting point of 80° C. or below is ibuprofen, dexibuprofen (S(+)-ibuprofen) or a mixture thereof.

4. The fused solid dispersion of claim 1, which further comprises a tabletting aid selected from the group consisting of a sugar alcohol, a water soluble polymer, an oily base and a mixture thereof.

5. The fused solid dispersion of claim 4, which comprises the active ingredient and the tabletting aid in a weight ratio ranging 1:0 to 1:2.

6. The fused solid dispersion of claim 1, wherein the pharmaceutically acceptable absorbent is selected from the group consisting of light anhydrous silicic acid, hydrotalcite, aluminum magnesium silicate, aluminum hydroxide, aluminum silicate, magnesium aluminum methasilicate, bentonite, lactose, dextrin, starch, microcrystalline cellulose, hydroxypropylmethyl cellulose, hydroxypropyl cellulose, hydroxyethyl cellulose, ethyl cellulose, methyl cellulose, polyethylene glycol, finely-divided cross-linked polyvinylpyrrolidone and a mixture thereof.

7. The fused solid dispersion of claim 4, wherein the sugar alcohol is selected from the group consisting of xylitol, sorbitol, mannitol and a mixture thereof.

8. The fused solid dispersion of claim 4, wherein the water soluble polymer is selected from the group consisting of hydroxypropylmethyl cellulose, hydroxypropyl cellulose, polyvinylpyrrolidone, polyethylene glycol, polyethylene oxide, polyvinyl alcohol and a mixture thereof.

9. The fused solid dispersion of claim 4, wherein the oily base is selected from the group consisting of sucrose fatty acid ester, glyceryl behenate, glyceryl palmitostearate, glyceryl monooleate, glyceryl monostearate and a mixture thereof.

10. A tablet for oral administration comprising the fused solid dispersion according to claim 1.

11. The tablet for oral administration of claim 10, which is a controlled release tablet comprising the fused solid dispersion and a release-controlling agent for the slow release of the active ingredient.

12. The tablet for oral administration of claim 11, which further comprises a pharmaceutically acceptable excipient.

13. The tablet for oral administration of claim 12, which comprises the fused solid dispersion, the release-controlling agent and the pharmaceutically acceptable excipient in a weight ratio ranging from 1:0.01˜3:0˜3.

14. The tablet for oral administration of claim 10, which is a multilayer tablet consisting of a rapid release layer comprising the fused solid dispersion and the pharmaceutically acceptable excipient, and a controlled release layer comprising the fused solid dispersion and a release-controlling agent.

15. The tablet for oral administration of claim 11, wherein the release-controlling agent is selected from the group consisting of polyethylene oxide having a molecular weight ranging from 10,000 to 9,000,000, hydroxypropylmethyl cellulose having a molecular weight ranging from 1,000 to 4,000,000, hydroxypropyl cellulose, carboxyvinyl polymer, polyvinyl alcohol, xanthan gum, guar gum, locust bean gum, carboxymethyl cellulose and its derivative, methyl cellulose and its derivative, and povidone-polyvinylacetate copolymer having a molecular weight ranging from 2,000 to 2,000,000.

16. The tablet for oral administration of claim 14, wherein the controlled release layer further comprises a pharmaceutically acceptable excipient.

17. The tablet for oral administration of claim 12, wherein the pharmaceutically acceptable excipient is selected from the group consisting of a cross-linked polyvinylpyrrolidone, a cross-linked sodium carboxymethyl cellulose, carboxymethyl starch, calcium methacrylate-divinylbenzene copolymer, polyvinyl alcohol, lactose, microcrystalline cellulose and cellulose derivative, starch and its derivative, cyclodextrin and dextrin derivative, pregelatinized starch and its derivative, colloidal silica, magnesium stearate, glyceryl monostearate, sodium stearyl fumarate, and hydrogenated caster oil.

18. The tablet for oral administration of claim 14, wherein the rapid release layer comprises the fused solid dispersion and the pharmaceutically acceptable excipient in a weight ratio ranging from 1:0.05 to 1:3.

19. The tablet for oral administration of claim 16, wherein the controlled release layer comprises the fused solid dispersion, the release-controlling agent and the pharmaceutically acceptable excipient in a weight ratio ranging from 1:0.01˜3:0˜3.

20. A process for preparing the tablet for oral administration of claim 10 comprising:
(a) heating to melt an active ingredient having a melting point of 80° C. or below and adding a pharmaceutically acceptable absorbent having a specific surface area ranging from 20 to 400 m²/g thereto to obtain a homogenous fused solid dispersion;
(b) cooling, drying and pulverizing the fused solid dispersion obtained in step (a) to obtain granules; and
(c) adding a release-controlling agent or a pharmaceutically acceptable excipient to the granules obtained in step (b) and compressing the resulting mixture into a tablet.

21. The process of claim 20, which further comprises the step of adding a tabletting aid selected from the group consisting of a sugar alcohol, a water soluble polymer, an oily base and a mixture thereof, when adding the pharmaceutically acceptable absorbent in step (a).

22. The tablet for oral administration of claim 14, wherein the release-controlling agent is selected from the group consisting of polyethylene oxide having a molecular weight ranging from 10,000 to 9,000,000, hydroxypropylmethyl cellulose having a molecular weight ranging from 1,000 to 4,000,000, hydroxypropyl cellulose, carboxyvinyl polymer, polyvinyl alcohol, xanthan gum, guar gum, locust bean gum, carboxymethyl cellulose and its derivative, methyl cellulose and its derivative, and povidone-polyvinylacetate copolymer having a molecular weight ranging from 2,000 to 2,000,000.

23. The tablet for oral administration of claim 14, wherein the pharmaceutically acceptable excipient is selected from the group consisting of a cross-linked polyvinylpyrrolidone, a cross-linked sodium carboxymethyl cellulose, carboxymethyl starch, calcium methacrylate-divinylbenzene copolymer, polyvinyl alcohol, lactose, microcrystalline cellulose and cellulose derivative, starch and its derivative, cyclodextrin and dextrin derivative, pregelatinized starch and its derivative, colloidal silica, magnesium stearate, glyceryl monostearate, sodium stearyl fumarate, and hydrogenated caster oil.

24. The tablet for oral administration of claim 16, wherein the pharmaceutically acceptable excipient is selected from the group consisting of a cross-linked polyvinylpyrrolidone, a cross-linked sodium carboxymethyl cellulose, carboxymethyl starch, calcium methacrylate-divinylbenzene copolymer, polyvinyl alcohol, lactose, microcrystalline cellulose and cellulose derivative, starch and its derivative, cyclodextrin and dextrin derivative, pregelatinized starch and its derivative, colloidal silica, magnesium stearate, glyceryl monostearate, sodium stearyl fumarate, and hydrogenated caster oil.
